(19) United States
(12) Patent Application Publication
(10) Pub. No.: US 2017/0304902 A1
(43) Pub. Date: Oct. 26, 2017

(54) SYNTHESIS OF WATER-SOLUBLE THIOLATE-PROTECTED GOLD NANOPARTICLES OF UNIFORM SIZE AND CONJUGATES THEREOF

(71) Applicant: The Board of Trustees of the Leland Stanford Junior University, Stanford, CA (US)

(72) Inventors: Maia Azubel, Menlo Park, CA (US); Roger D. Kornberg, Atherton, CA (US)

(21) Appl. No.: 15/493,726

(22) Filed: Apr. 21, 2017

Related U.S. Application Data
(60) Provisional application No. 62/326,559, filed on Apr. 22, 2016.

(51) Int. Cl. B22F 9/24; A61K 49/04; A61K 49/22; C07K 16/40

(52) U.S. Cl. CPC B22F 9/24; A61K 49/222; C07K 16/40; A61K 49/04; B22F 2009/245; B22F 2998/10; C07K 2317/622; B22F 2301/255

(57) ABSTRACT

Methods of synthesizing water-soluble thiolate-protected gold nanoparticles of uniform size and conjugates thereof are disclosed. In particular, the invention relates to a method of synthesizing homogeneous, water-soluble gold nanoparticles by using a modified Brust procedure and methods of conjugating them. Gold nanoparticles, produced by the methods of the invention, are useful in various therapeutic and imaging applications where the use of gold nanoparticles having uniform structural and optical properties is desired.

### CROSS-REFERENCE TO RELATED APPLICATION

This application claims benefit under 35 U.S.C. §119(e) of provisional application 62/326,559, filed Apr. 22, 2016, which application is hereby incorporated by reference in its entirety.

### STATEMENT REGARDING FEDERALLY SPONSORED RESEARCH OR DEVELOPMENT

This invention was made with Government support under contract AI021144 awarded by the National Institutes of Health. The Government has certain rights in the invention.

### TECHNICAL FIELD

The present invention pertains generally to methods of synthesizing gold nanoparticles of uniform size and conjugates thereof and their use in therapeutics and imaging.

### BACKGROUND

Following the first syntheses by Brust et al. (J. Chem. Soc., Chem. Commun. (1994) 801-802; J. Chem. Soc., Chem. Commun. (1995) 1655-1656), many thiolate monolayer-protected gold nanoparticles have been described. The nanoparticles are usually heterogeneous and variable in size from one preparation to another. Most syntheses have been performed in partial or fully organic systems, yielding organo-soluble particles (Zaluzhna et al. (2012) Chem. Commun. (Camb). 48(3):362-364, Li et al. (2011) Chem. Commun. (Camb) 47(21):6033-6035, Yee et al. (1999) Langmuir 15 (10):3486-3491). The first water-soluble, thiolate monolayer-protected, gold nanoparticles were synthesized with a mixture of methanol and water as solvents (Schaaff et al. (1998) J. Phys. Chem. B 102 (52):10643-10646). Synthesis of water-soluble particles was later extended to a wide range of thiols in fully aqueous systems (Ackerson et al. (2005) J. Am. Chem. Soc. 127(18):6550-6551, Ackerson et al. (2010) Bioconjug. Chem. 21(2):214-218, Wong et al. (2015) ACS Comb. Sci. 17(1):11-18).

Synthesis is accomplished in two steps, reduction of Au⁺³ to Au⁺ by thiol, and further reduction to Au⁰ by borohydride. The ratio of thiol to Au influences the nanoparticle size, with decreasing ratio reported to favor larger sizes (Hostetler et al. (1998) Langmuir 14:17-30). Following synthesis, surface thiols may be replaced by others in a reaction referred to as Murray place exchange (Hostetler et al. (1999) Langmuir 15:3782-3789).

For many applications (Rosi et al. (2006) Science 312:1027-1030, Qian et al. (2008) Nat. Biotechnol. 26(1):83-90, Daniel et al. (2004) Chem. Rev. 104(1):293-346), nanoparticles of uniform size and controlled reactivity are required. Water-soluble particles are of particular interest in life science and medicine. Previous studies have yielded nanoparticles heterogeneous in size.

### SUMMARY

The present invention is based on the development of a method for synthesizing gold nanoparticles of uniform size and conjugates thereof. In particular, the invention relates to a method of synthesizing homogeneous, water-soluble gold nanoparticles by using a modified Brust procedure and their use in various applications in science and medicine. Gold nanoparticles, produced by the methods of the invention, are useful in various therapeutic and imaging applications where the use of gold nanoparticles having uniform structural and optical properties is desired.

In one aspect, the invention includes a method of synthesizing gold nanoparticles, the method comprising: adding a thiol and chloroauric acid to a mixture of methanol and water, adjusting pH of the mixture to about 13-14, equilibrating the mixture for at least 14 hours, and reacting with borohydride, whereby gold nanoparticles of uniform size are produced. In one embodiment, the mixture is equilibrated for a time ranging from about 16 to about 20 hours prior to reacting with borohydride.

In another embodiment, the thiol is selected from the group consisting of 3-mercaptobenzoic acid (3-MBA), 4-mercaptobenzoic acid (4-MBA), thiomalate, glutathione, and N-acetyl-L-cysteine.

The size of the gold nanoparticles depends on the particular thiol that is used and the ratio of the thiol to gold in the mixture. In certain embodiments, the thiol and the chloroauric acid are added to the mixture at a thiol to gold ratio of 2:1, 3:1, 4:1, 5:1, 6:1, or 7:1, or any other ratio that produces gold nanoparticles of uniform size.

The gold nanoparticles produced by the methods described herein can be conjugated to various molecules useful in scientific or medical applications. For example, the gold nanoparticles can be conjugated to a therapeutic agent or a targeting agent. In certain embodiments, the gold nanoparticles are conjugated to one or more biomolecules such as, but not limited to, a nucleic acid (e.g., DNA or RNA), oligonucleotide (e.g., siRNA or probe), protein (e.g., enzyme, antibody, or receptor), peptide (e.g., ligand or antigen), carbohydrate, or lipid. Gold nanoparticles can also be conjugated to various other types of molecules, including but not limited to, drugs, polymers, fluorescent dyes, aptamers, and dendrimers.

In another aspect, the invention includes a composition comprising gold nanoparticles of uniform size produced by a method described herein. The composition may further comprise a pharmaceutically acceptable carrier. In one embodiment, the gold nanoparticles in the composition are conjugated to a molecule comprising a sulfhydryl group. In another embodiment, the gold nanoparticles in the composition are conjugated to a biomolecule. In yet another embodiment, the gold nanoparticles in the composition are conjugated to a therapeutic agent and/or a targeting agent.

In another aspect, the invention includes a method of treating a disease or disorder comprising administering a composition comprising gold nanoparticles of uniform size produced by a method described herein to a subject in need of treatment of the disease or disorder. In one embodiment, the gold nanoparticles are conjugated to a therapeutic agent for treating the subject for the disease or disorder. In another embodiment, the gold nanoparticles are conjugated to a therapeutic agent and a targeting agent, wherein the targeting agent localizes the gold nanoparticles to a site in need of treatment by the therapeutic agent.

In another aspect, the invention includes a method of imaging gold nanoparticles, the method comprising a) administering a composition comprising gold nanoparticles of uniform size, produced by a method described herein, to a subject, wherein the gold nanoparticles are conjugated to a targeting agent that localizes the gold nanoparticles to a site of interest in the subject; and b) obtaining an image of the gold nanoparticles. In certain embodiments, the gold nanoparticles are further conjugated to a therapeutic agent capable of treating a disease or disorder at the site of interest.

These and other embodiments of the subject invention will readily occur to those of skill in the art in view of the disclosure herein.

### BRIEF DESCRIPTION OF THE FIGURES

FIG. 1 shows the dependence of gold nanoparticle size upon the thiol-to-gold ratio. Nanoparticles were synthesized with 3-MBA at the ratio to HAuCl₄ indicated above the lanes and were analyzed by 10% glycerol, 12% PAGE.

FIGS. 2A-2C show transmission electron microscopy (TEM) of 3-MBA-protected gold nanoparticles. FIG. 2A shows a Cryo-EM image of particles synthesized with a 3-MBA-to-gold ratio of 2. FIG. 2B shows a Cryo-EM image of particles synthesized with a 3-MBA-to-gold ratio of 3. FIG. 2C shows room temperature EM image of particles synthesized with a 3-MBA-to-gold ratio of 7. Bar represents 10 nm.

FIG. 3 shows the importance of equilibration in the first step of gold nanoparticle synthesis and stability of the products. Synthesis of 3-MBA-protected gold nanoparticles was performed without (lane 1) or with (lanes 2 and 3) equilibration for about 16 hours before the addition of NaBH₄. Nanoparticles were analyzed by 10% glycerol, 12% PAGE immediately after synthesis (lanes 1 and 2) or following storage for 3.5 years at 4° C. (lane 3)

FIG. 4 shows that homogeneous gold nanoparticles formed with the thiols indicated at a thiol-to-gold ratio of 3. Analysis of the reaction products from different thiol-protected gold nanoparticles was performed with 10% glycerol, 12% PAGE.

FIG. 5 shows that exchange of 3-MBA for other thiols is irreversible. 3-MBA-protected gold nanoparticles were treated with 10 mM glutathione, DTNB (5,5′-dithiobis 2-nitrobenzoic acid), or 4-MBA, and then treated with 3-MBA (+) or not (−). Nanoparticles were analyzed by 10% glycerol, 12% PAGE

FIG. 6 shows reactivities of gold nanoparticles (AuNPs) towards a protein sulfhydryl. Nanoparticles formed with 3-MBA were subjected to exchange with glutathione, DTNB, 4-MBA, and N-acetyl-L-cysteine, or not (−). The nanoparticles were treated with a single chain antibody fragment bearing a surface-exposed cysteine residue. Nanoparticles and scFv-nanoparticle conjugates were analyzed by 10% glycerol, 12% PAGE. The band labeled with the symbol for an antibody fragment contained protein, revealed by staining with Coomassie Blue (not shown).

FIG. 7 shows that reproducibility is maintained after scaling up 3-MBA protected AuNPs syntheses. 3-MBA protected AuNPs were synthesized in small (5 ml), medium (100 ml) or large (500 ml) scale and analyzed by 12% PAGE.

FIG. 8 shows that the size of gold nanoparticles depends on the thiol-to-gold ratio and not the actual concentration of gold and thiol. Nanoparticles were synthesized at a variable (left) or constant (right) 4-MBA-to-HAuCl₄ ratio and analyzed by 12% PAGE.

FIGS. 9A and 9B show a comparison of the dependence of gold nanoparticle size upon the thiol-to-gold ratio for different thiols. Nanoparticles were synthesized with GSH (FIG. 9A) and 4-MBA (FIG. 9B) at the ratio of thiol to HAuCl₄ indicated above the lanes, and were analyzed by 12% PAGE.

FIG. 10 shows ligand exchange of 3-MBA for other thiols. 3-MBA protected AuNPs were treated with increasing concentrations of N-acetyl-L-cysteine, thiomalate, or SHEtNH₂ and analyzed by 12% PAGE.

FIG. 11 shows the reactivity of 3-MBA protected AuNPs towards a protein sulfhydryl. 3-MBA protected AuNPs were synthesized at 3 different thiol-to-gold ratios (2, 3, and 7). 3-MBA protected AuNPs were incubated with a scFv bearing a surface-exposed cysteine residue. Reaction products were analyzed by 12% SDS PAGE, unstained (top panel) or stained with Coomassie blue (bottom panel). Left lane, precision plus protein standards (BioRad). Second lane from left, unlabeled scFv.

FIG. 12 shows that the reactivity of a sulfhydryl at the 3′-end of an oligodeoxynucleotide is greater towards a 3-MBA protected AuNP than towards a 4-MBA protected AuNP. 4-MBA or 3-MBA protected AuNPs were incubated with a 3′-end SH-modified oligodeoxynucleotide (oligo) (+) or not (−) and analyzed by 12% PAGE. Diagrams to the right (for 4-MBA) and left (for 3-MBA): ball indicates free AuNPs; ball with 1, 2, 3 or 4 bars indicate AuNPs conjugated to 1, 2, 3 or 4, respectively, molecules of DNA.

FIG. 13 shows that gold conjugation has no adverse effect on annealing, and boiling has no adverse effect on stability of Au-DNA conjugates. 1:1 Au:DNA conjugates of complementary sequence were annealed by boiling and slowly decreasing the temperature to 25° C. Conjugates before (+oligoA, +oligoB) and after annealing were analyzed by 12% PAGE.

### DETAILED DESCRIPTION OF THE INVENTION

The practice of the present invention will employ, unless otherwise indicated, conventional methods of chemistry, biochemistry, molecular biology and recombinant DNA techniques, medicine, and immunology, within the skill of the art. Such techniques are explained fully in the literature. See, e.g., Gold Nanoparticles: Properties, Characterization and Fabrication (Nanotechnology Science and Technology, P. E. Chow, ed., Nova Science Publishers, Inc., 2010); Gold Nanoparticles: Synthesis, Optical Properties and Applications for Cancer Treatment (Nanotechnology Science and Technology, A. Jarnagin and L. Halshauser eds., Nova Science Publishers, Inc., 2013); C. Louis and O. Pluchery Gold Nanoparticles for Physics, Chemistry and Biology (Imperial College Press, 2012); Caister Academic Press, 1^{st} edition, 2010; Nanomedicine ((Frontiers of Nanoscience, H. D. Summers, Elsevier, 2013); Handbook of Experimental Immunology, Vols. I-IV (D. M. Weir and C. C. Blackwell eds., Blackwell Scientific Publications); T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (3^{rd} Edition, 2001); Methods In Enzymology (S. Colowick and N. Kaplan eds., Academic Press, Inc.).

All publications, patents and patent applications cited herein, whether supra or infra, are hereby incorporated by reference in their entireties.

### 1. DEFINITIONS

In describing the present invention, the following terms will be employed, and are intended to be defined as indicated below.

It must be noted that, as used in this specification and the appended claims, the singular forms “a,” “an” and “the” include plural referents unless the content clearly dictates otherwise. Thus, for example, reference to “a nanoparticle” includes a mixture of two or more such nanoparticles, and the like.

As used herein, “about” or “approximately” mean within 20 percent, preferably within 10 percent, and more preferably within 5 percent of a given value or range.

“Substantially purified” generally refers to isolation of a substance (compound, polynucleotide, oligonucleotide, protein, or polypeptide) such that the substance comprises the majority percent of the sample in which it resides. Typically in a sample, a substantially purified component comprises 50%, preferably 80%-85%, more preferably 90-95% of the sample. Techniques for purifying polynucleotides oligonucleotides and polypeptides of interest are well-known in the art and include, for example, ion-exchange chromatography, affinity chromatography and sedimentation according to density.

By “isolated” is meant, when referring to a polypeptide, that the indicated molecule is separate and discrete from the whole organism with which the molecule is found in nature or is present in the substantial absence of other biological macro-molecules of the same type. The term “isolated” with respect to a polynucleotide or oligonucleotide is a nucleic acid molecule devoid, in whole or part, of sequences normally associated with it in nature; or a sequence, as it exists in nature, but having heterologous sequences in association therewith; or a molecule disassociated from the chromosome.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably.

Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

The terms “polypeptide” and “protein” refer to a polymer of amino acid residues and are not limited to a minimum length. Thus, peptides, oligopeptides, dimers, multimers, and the like, are included within the definition. Both full-length proteins and fragments thereof are encompassed by the definition. The terms also include postexpression modifications of the polypeptide, for example, glycosylation, acetylation, phosphorylation, hydroxylation, oxidation, and the like.

The term “antibody” encompasses polyclonal and monoclonal antibody preparations, as well as preparations including hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, e.g., Huston et al. (1988) Proc Natl Acad Sci USA 85:5879-5883); dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

The term “subject” includes both vertebrates and invertebrates, including, without limitation, mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, geese, and the like.

“Treatment” of a subject or “treating” a subject for a disease or condition herein means reducing or alleviating clinical symptoms of the disease or condition.

### 2. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery of a method for synthesizing water-soluble gold nanoparticles of uniform size by using a modified Brust procedure (Brust, M.; Walker, M.; Bethell, D.; Schiffrin, D. J.; Whyman, R. Journal of the Chemical Society, Chemical Communications 1994, 801; Brust, M.; Fink, J.; Bethell, D.; Schiffrin, D. J.; Kiely, C. Journal of the Chemical Society, Chemical Communications 1995, 1655; herein incorporated by reference in their entireties). Synthesis of the gold nanoparticles comprises a first step in which the Au⁺³ is reduced to Au⁺ by a thiol, and a second step in which Au⁺ is further reduced to Au⁰ by a borohydride. The inventors have shown that gold nanoparticles uniform in size can be synthesized by equilibration of a chloroauric acid-thiol solution at about pH 13-14 for approximately 14-20 hours prior to reduction by borohydride (Example 1). Gold nanoparticles, produced by the methods of the invention, are useful in various therapeutic and imaging applications where the use of gold nanoparticles having uniform structural and optical properties is desired.

Exemplary thiol reagents that can be used in the practice of the invention include 3-mercaptobenzoic acid (3-MBA), 4-mercaptobenzoic acid (4-MBA), thiomalate, glutathione, and N-acetyl-L-cysteine. The choice of thiol affects the reactivity and size of the gold nanoparticles that are produced. In particular, the size of the gold nanoparticles is dependent on the ratio of the thiol to gold in the reaction mixture. Gold nanoparticles larger in size can be produced by increasing the thiol to gold ratio. Accordingly, the thiol to gold ratio can be adjusted to produce nanoparticles of a desired size. In certain embodiments, the thiol and the chloroauric acid are added to a reaction mixture at a thiol to gold ratio of 2:1, 3:1, 4:1, 5:1, 6:1, or 7:1, or any other ratio that produces gold nanoparticles of uniform size at a desired size.

Thiols on the surface of the gold nanoparticles, so produced, may be exchanged with other thiols by carrying out a Murray place exchange reaction (Hostetler, M. J.; Templeton, A. C.; Murray, R. W. Langmuir 1999, 15, 3782; herein incorporated by reference in its entirety). Place-exchange reactions can be used for preparing conjugates of the gold nanoparticles with any molecule comprising a sulfhydryl group. For example, biomolecules that contain a thiol group naturally (e.g., protein containing a surface-exposed cysteine) may be conjugated to the gold nanoparticles. Alternatively, a biomolecule may be derivatized to add a thiol functional group (e.g., thiol-modified oligonucleotide, polypeptide, or carbohydrate) to allow conjugation to the gold nanoparticles.

In certain embodiments, the gold nanoparticles are conjugated to one or more biomolecules, such as, but not limited to, nucleic acids (e.g., DNA or RNA)), oligonucleotides (e.g., probes or siRNA), proteins (e.g., enzymes, antibodies, or receptors), peptides (e.g., ligands or antigens), carbohydrates (e.g., lactose, glucose, or mannose), or lipids. Gold nanoparticles can also be conjugated to various other types of molecules, including, but not limited to, drugs, polymers, fluorescent dyes, aptamers, or dendrimers.

In certain embodiments, the gold nanoparticles are conjugated to a targeting agent such as a peptide comprising a membrane translocation signal that is capable of transporting a gold nanoparticle across a cell membrane, a peptide comprising a localization signal that can be used for intracellular targeting, or a homing peptide that can be used for targeting specific organs, tissues, or cells. Targeting peptides may comprise a targeting sequence, including, but not limited to a secretory protein signal sequence, a membrane protein signal sequence, a nuclear localization sequence, a nucleolar localization signal sequence, an endoplasmic reticulum localization sequence, a peroxisome localization sequence, a mitochondrial localization sequence, and a protein-protein interaction motif sequence. Targeting agents may include homing peptides that recognize tissue-specific markers, organ-specific markers, or disease-specific markers (e.g., cell surface epitope associated with a specific disease state or tumor marker). Exemplary targeting agents include an RGD peptide, an NGR peptide, folate, transferrin, GM-CSF, galactosamine, growth factor receptors (e.g. IGF-1R, MET, EGFR), antibodies and antibody fragments including anti-VEGFR, anti-ERBB2, anti-tenascin, anti-CEA, anti-MUC1, anti-TAG72, mutagenic bacterial strain markers, and fatty acids. Targeting agents may also comprise cell penetrating peptides (CPPs) capable of translocating a gold nanoparticle into a cell. Exemplary CPPs include HIV-Tat, penetratin, transportan, octaarginine, nonaarginine, antennapedia, TP10, Buforin II, MAP (model amphipathic peptide), K-FGF, Ku70, mellittin, pVEC, Pep-1, SynB1, Pep-7, CADY, GALA, pHLIP, KALA, R7W, and HN-1. In addition, CPPs may be cell-type specific, such as F3 which is capable of internalizing gold nanoparticles into tumor cells and blood, and LyP-1, which is capable of internalizing gold nanoparticles into lymphatic endothelial cells in tumors. For a description of various targeting agents, see, e.g., Laakkonen et al. (2010) Integr Biol (Camb) 2(7-8):326-37; Jones et al. (2012) J Control Release 161(2):582-591; Fonseca et al. (2009) Adv. Drug Deliv. Rev. 61(11):953-64; Schwarze et al. (1999) Science. 285(5433):1569-72; Derossi et al. (1996) J. Biol. Chem. 271(30):18188-18193; Fuchs et al. (2004) Biochemistry 43(9):2438-2444; and Yuan et al. (2002) Cancer Res. 62(15):4186-4190; herein incorporated by reference in their entireties.

In certain embodiments, the gold nanoparticles are conjugated to a therapeutic agent, such as a biomolecule or drug capable of treating a disease or disorder. Gold nanoparticles carrying a combination of a targeting agent and a therapeutic agent can be used for controlled drug delivery, wherein the targeting agent localizes the gold nanoparticles to a site in need of treatment (e.g., organ, tissue, cell-type, diseased or damaged tissue, tumor, or intracellular location) by the therapeutic agent. It will be understood by those of skill in the art that various targeting agents and/or therapeutic agents can be selected for conjugation to gold nanoparticles.

In addition, compositions comprising gold nanoparticles of uniform size are useful for imaging. The gold nanoparticles exhibit surface plasmon resonance (LSPR) with absorption and emission peaks within the visible range of light. Their properties make them useful in a variety of imaging techniques as contrast agents or for electric field enhancement. In particular, gold nanoparticles can be used as contrast agents for biomedical imaging, including computed tomography (CT), photoacoustic (PA) imaging, and ultrasound imaging, as well as microscopy, including transmission electron microscopy (TEM), scanning transmission electron microscopy (STEM), photothermal microscopy, and plasmon coupling microscopy; and as electric field enhancers of Raman signals for surface enhanced Raman spectroscopy (SERS). In addition, gold nanoparticles can serve as carriers to deliver fluorescent dyes, bioluminescent proteins, or other light producing molecules for photoimaging. See, e.g., Ashton et al. (2015) Front Pharmacol. 6:256; Pekkanen et al. (2014) J Biomed Nanotechnol. 10(9):1677-712; Cole et al. (2015) Nanomedicine (Lond) 10(2):321-341; Li et al. (2015) Nanomedicine (Lond) 10(2):299-320; Mayhew et al. (2015) Cell Tissue Res. 360(1):43-59; Peng et al. (2015) Anal Chem. 87(1):200-215; Guo et al. (2014) Bioconjug Chem 25(5):840-854; Vermeulen et al. (2014) J Microsc. 254(3):115-121; Turzhitsky et al. (2014) Appl Spectrosc 68(2):133-154; Curry et al. (2014) Contrast Media Mol Imaging. 9(1):53-61; Peckys et al. (2014) Microsc Microanal. 20(2):346-365; and Wu et al. (2014) Chem Soc Rev. 43(11):3884-3897; herein incorporated by reference.

In particular, compositions comprising gold nanoparticles of uniform size, produced as described herein can be used for in vivo imaging of cells and tissue. In certain embodiments, the gold nanoparticles are conjugated to a targeting agent that localizes the gold nanoparticles to a site of interest (e.g., site of diseased or damaged tissue) in a subject to allow imaging of the gold nanoparticles at the site of interest. Preferably, a detectably effective amount of the gold nanoparticles is administered to a subject; that is, an amount that is sufficient to yield an acceptable image using the imaging equipment that is available for clinical use. A detectably effective amount of a composition comprising gold nanoparticles may be administered in more than one injection if needed. The detectably effective amount of the gold nanoparticles needed for an individual may vary according to factors such as the age, sex, and weight of the individual, and the particular medical imaging device used. Optimization of such factors is within the level of skill in the art.

The gold nanoparticles may be further conjugated to a therapeutic agent to produce a theranostic agent capable of both imaging and treating a disease or disorder at a site of interest. Imaging with such gold nanoparticle theranostic agents can be used in assessing efficacy of therapeutic drugs in treating a disease or disorder. For example, images can be acquired after treatment with a gold nanoparticle theranostic agent to determine if the individual is responding to treatment. In a subject with cancer, imaging with a gold nanoparticle-targeted theranostic agent can be used to evaluate whether a tumor is shrinking or growing. Further, the extent of cancerous disease (stage of cancer progression) can be determined to aid in determining prognosis and evaluating optimal strategies for treatment (e.g., surgery, radiation, or chemotherapy).

### 3. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Synthesis of Water-Soluble, Thiolate-Protected Gold Nanoparticles, Uniform in Size

Introduction

By a modification of the method of Brust et al., water-soluble, thiolate-protected gold nanoparticles, uniform in size, were synthesized, with no requirement for purification. The modification of the method was equilibration in the first step, which proved crucial for achieving size homogeneity. The thiol-to-gold ratio controlled the size of the particles, and the choice of thiol controlled the reactivity of the particles towards thiol exchange.

Experimental Section

Synthesis

The thiols 3-mercaptobenzoic acid (3-MBA), 4-mercaptobenzoic acid (4-MBA), thiomalate, and N-acetyl-L-cysteine, and HAuCl₄, were from Sigma-Aldrich. Glutathione (GSH) was from EMD. Thiols (84 mM) and HAuCl₄ (28 mM) were dissolved in methanol immediately before use and mixed the ratios indicated. Water (2.5 vol) was added and the pH was adjusted to 13 with NaOH to dissolve insoluble material. The mixture was equilibrated for 16 hours at room temperature with mixing by rotation, during which the solution changed from yellow to colorless. Methanol and water were added to obtain a solution of 2.5 mM thiol in 27% (v/v) methanol. NaBH₄, freshly dissolved in water at 150 mM, was added to a final concentration of 2 mM and allowed to react for 4.5 hours at room temperature on a rocking platform. The reaction was stopped and the product precipitated by adjustment of the NaCl to 100 mM and by the addition of 2 volumes of methanol. The precipitate was collected by centrifugation for 10 minutes at 5,000 rpm, washed with 75% methanol, dried in air overnight, and resuspended in water. A reaction mixture of 100 ml yielded 5-10 mg of nanoparticles, soluble at millimolar concentrations in water, and stable at 4° C. for over 48 months, or at room temperature in dry form. Nanoparticles were analyzed in 10% glycerol, 12% polyacrylamide gels in Tris-borate-EDTA buffer at 150 volts.

Transmission Electron Microscopy (TEM)

For TEM at room temperature, nanoparticles (2 μl of 0.08 mg/ml in 175 mM KCl) were applied to a glow discharged, 400 mesh, ultrathin carbon film/holey carbon copper grid (Ted Pella, Inc.) for 30 seconds and blotted from the side. For cryo-TEM, nanoparticles (3 μl of 1 mg/ml) were applied to a glow discharged 200 mesh, Lacey carbon copper grid (SPI supplies) and frozen with a Vitrobot Mark V (FEI). Dried and frozen-hydrated samples were imaged under low-dose conditions (about 10 e−/Å²) at a magnification of 80,000 and defocus ranging from −0.2 to −1.2 μm, on an FEI (Eindhoven, The Netherlands) Tecnai F20 FEG transmission electron microscope operating at 200 kV and equipped with a 4K×4K CCD camera (Gatan US4000).

Ligand Exchange

Nanoparticles prepared with 3-MBA (1 μl of 0.5 mM) were treated with 20 μl of 10 mM GSH, 10 mM 5,5′-dithiobis-2-nitrobenzoic acid, 10 mM 4-MBA, 10-1000 mM N-Acetyl-L-cysteine, 10-1000 mM thiomalate, or 1-1000 mM cysteamine (SHEtNH₂) for 1 hour at 37° C. Reverse reactions were performed with 500 mM 3-MBA for 1 hour at 37° C. Products were analyzed in 10% glycerol, 12% polyacrylamide gels in Tris-borate-EDTA buffer at 150 volts.

Bioconjugation

The oligodeoxyribonucleotide 5′-CA GAT ATA TAA ATG CAA AAA CTG CAT AAC CAC TTT AAC TAA TAC TTT CAA/3ThioMC3-D/3′ (SEQ ID NO:1) and its complement (also 3ThioMC3-D-modified, from Integrated DNA Technologies) were treated at 500 μM in 10 mM Tris, pH 8, 1 mM EDTA with the reductant tris(2-carboxyethyl)phosphine (2 mM) for 1 hour at 37° C. The reduced oligodeoxyribonucleotide (4 μl of 25-200 μM) was allowed to react with 3-MBA or 4-MBA protected nanoparticles (1 μl of 0.5 mM) for 1 hour at 37° C. Products were analyzed in a 10% glycerol, 12% polyacrylamide gel in Tris-borate-EDTA buffer at 150 volts. For the isolation of the 1:1 conjugate, the gel band was excised, crushed and soaked overnight in water. Oligodeoxyribonucleotides were annealed by boiling and gradual cooling to 25° C.

A single chain antibody fragment (1 mg/ml) directed against RNA polymerase II, containing a surface-exposed cysteine, was reduced by treatment with 2 mM TCEP for 1 hour at 37° C. The reduced antibody fragment (2 μl) was allowed to react with 3-MBA (2 μl of 125-500 μM) for 1 hour at 37° C. Products were analyzed in a 10% glycerol, 12% SDS-polyacrylamide gel at 150 volts.

Results and Discussion

Homogeneous Nanoparticles

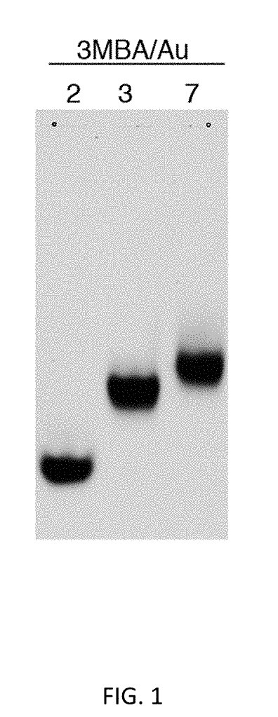

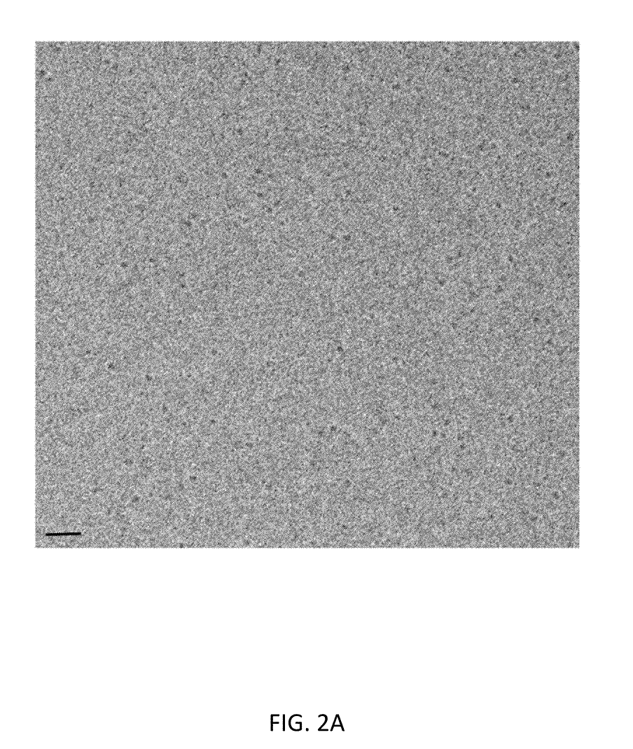

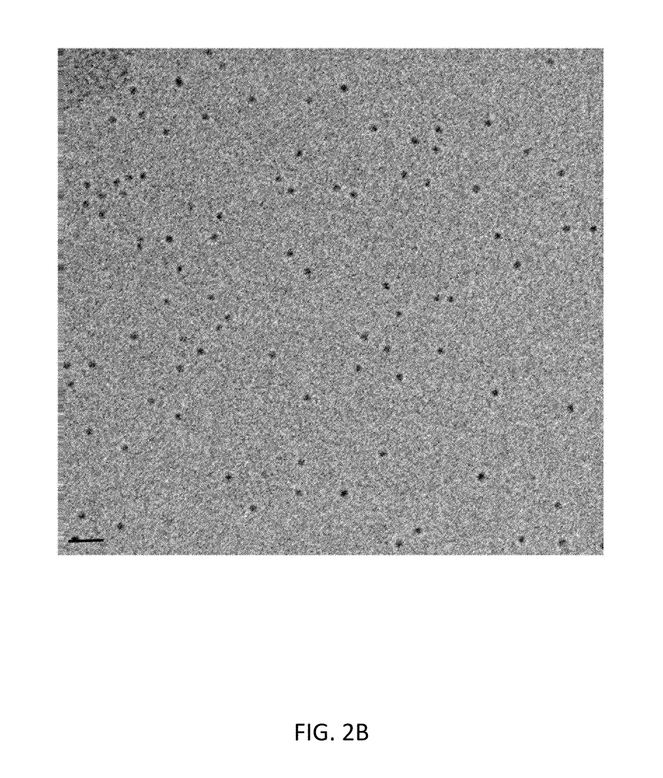

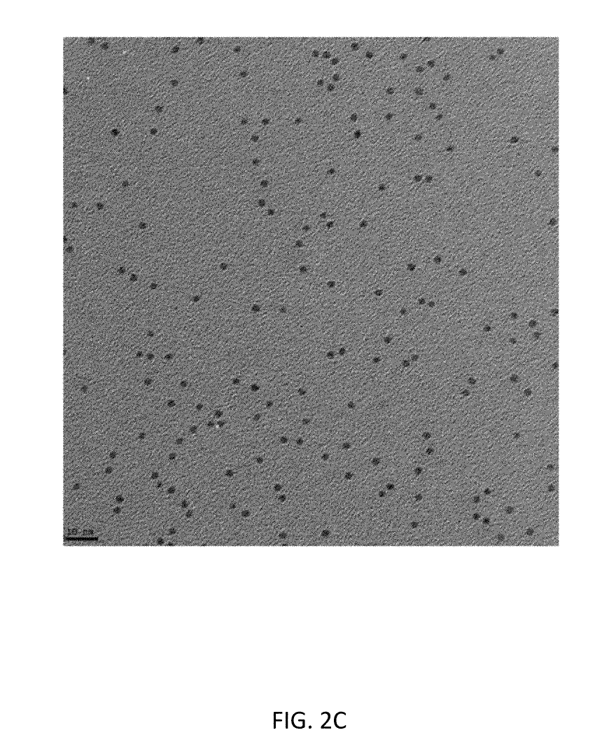

Following the method of the Brust et al. (J. Chem. Soc., Chem. Commun. (1994) 801-802), HAuCl₄ and thiol were dissolved in methanol and mixed, leading to the production of a white precipitate, which dissolved when the pH was adjusted to 13. The reaction was allowed to proceed for about 16 hours at room temperature, during which time the solution changed from yellow to colorless. Reduction was then performed with NaBH₄ for 4.5 hours at room temperature, and the product was precipitated with methanol and resuspended in water. The resulting particles appeared uniform in size by gel electrophoresis (FIG. 1) and electron microscopy (FIG. 2). Direct measurement of particle diameters from electron micrographs is, however, only approximate. Perfect uniformity can only be established by structure determination. We previously analyzed the smallest particles reported here by aberration-corrected electron microscopy and image processing, resulting in an electron density map at atomic resolution, showing that the particles contain 68 gold atoms (Azubel et al. (2014) Science 345: 909-912).

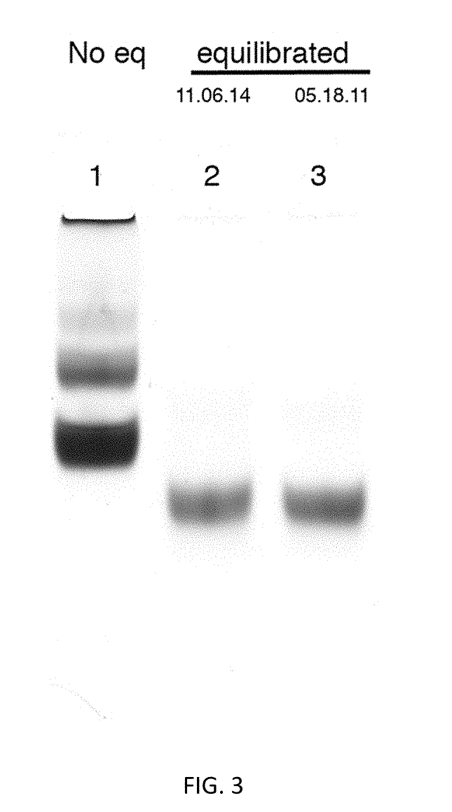

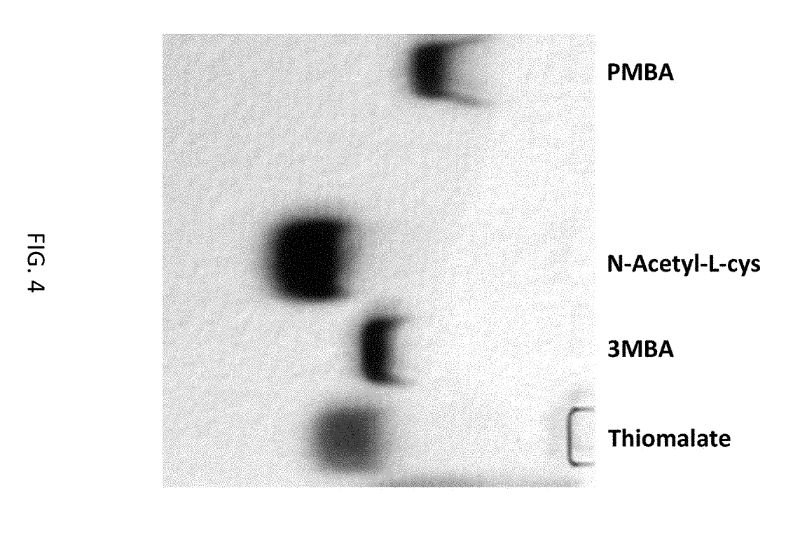

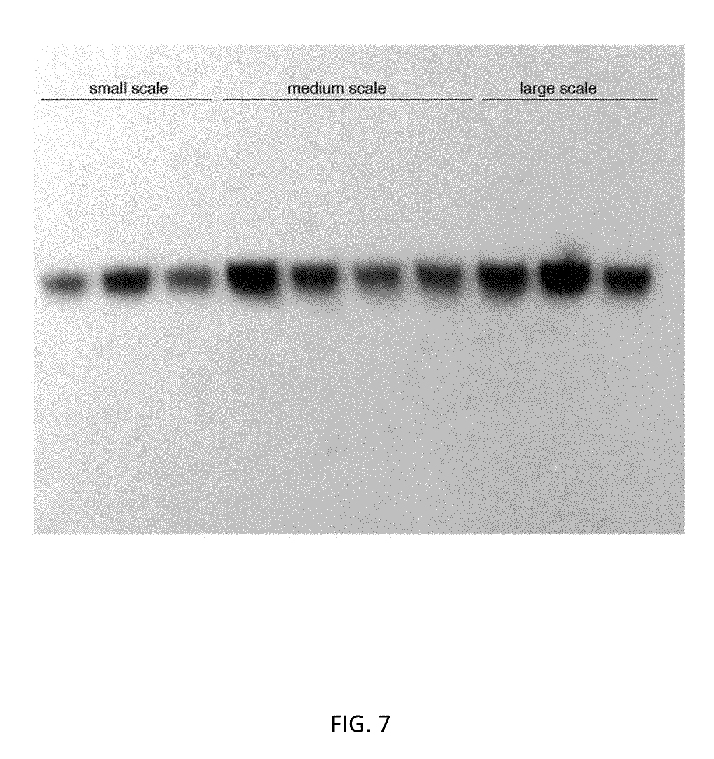

Two factors were critical for obtaining such uniformity, adjustment of the pH to 13, and equilibration for about 16 hours with the HAuCl₄-thiol solution (FIG. 3). Solutions of the same concentrations, prepared the same way, failed to produce uniform nanoparticles when the equilibration step was omitted (FIG. 3 lane 1). The thiol could be varied, with uniform particles obtained from 3-MBA, 4-MBA, thiomalate, glutathione, and N-acetyl-L-cysteine (FIG. 4). Syntheses could be increased in scale at least 100-fold with no decrease in yield (˜97%) or loss of homogeneity of the product (FIG. 7). The syntheses were highly reproducible and the products were stable for years in water.

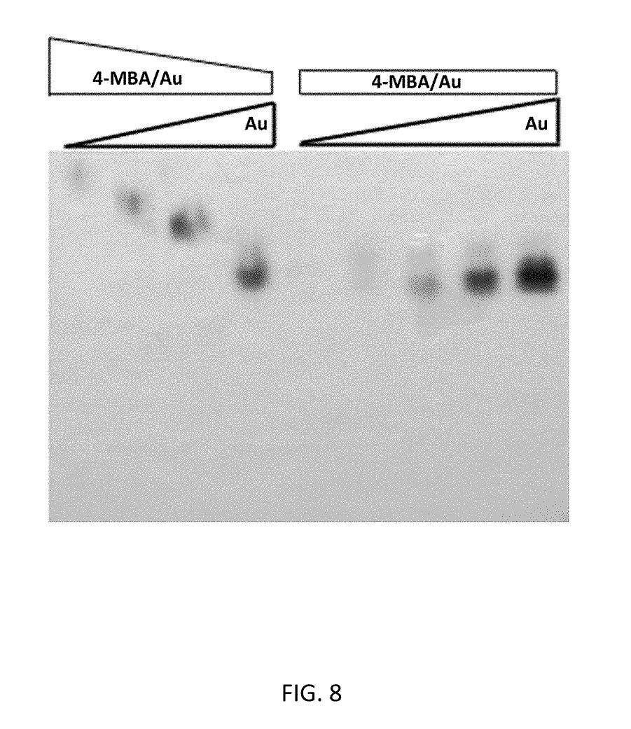

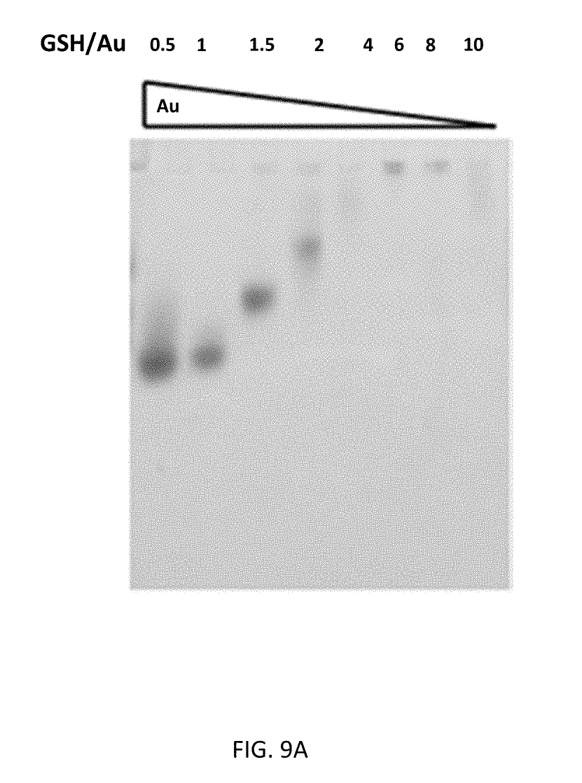

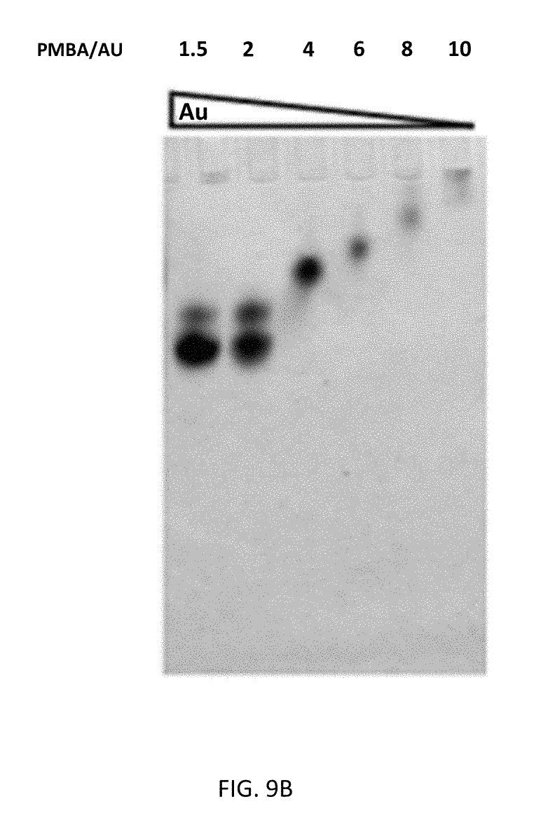

The size of the particles depended on the thiol-to-gold ratio. Only the ratio and not the actual concentrations of thiol and gold were important in the range tested (FIG. 8). Optimal ratios for the production of uniform particles depended on the thiol, and most often, though not always, a ratio of three or greater was required. Among the thiols tested, glutathione gave uniform particles at the smallest ratios (FIG. 9). In the case of 3-MBA, the smallest homogeneous particles were obtained at a ratio of two, larger homogeneous particles with three, and the largest homogeneous particles with a ratio of seven (FIG. 1).

Reactions of Nanoparticles

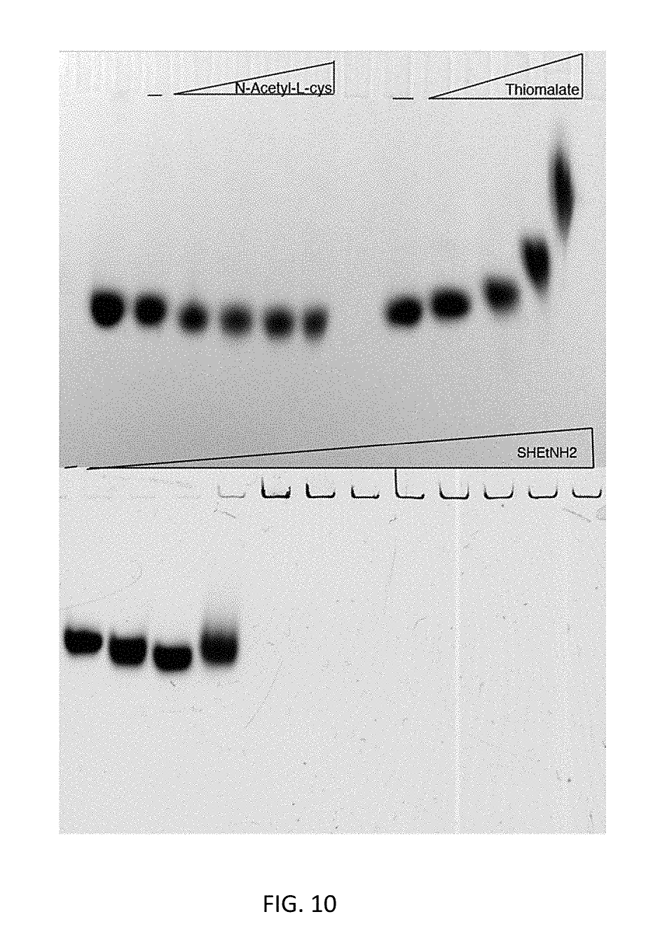

We employed the Murray Place Exchange reaction for ligand exchange, for conjugation to proteins bearing surface-exposed cysteine residues, and for conjugation to oligonucleotides bearing a sulfhydryl group at the 3′-end. For example, 3-MBA-protected particles were treated with increasing concentrations of various thiols at 37° C. for 1 hour, and changes in electrophoretic mobility were observed (FIG. 10). Such changes could be due to ligand exchange, to alteration of the gold core, or to a combination of the two. Several observations suggest that ligand exchange occurred without effect on the gold core: the particles remained uniform, as judged by electrophoresis, and in one case, conservation of the gold core was confirmed by X-ray crystallography (Heinecke et al. (2012) J. Am. Chem. Soc. 134(32):13316-13322).

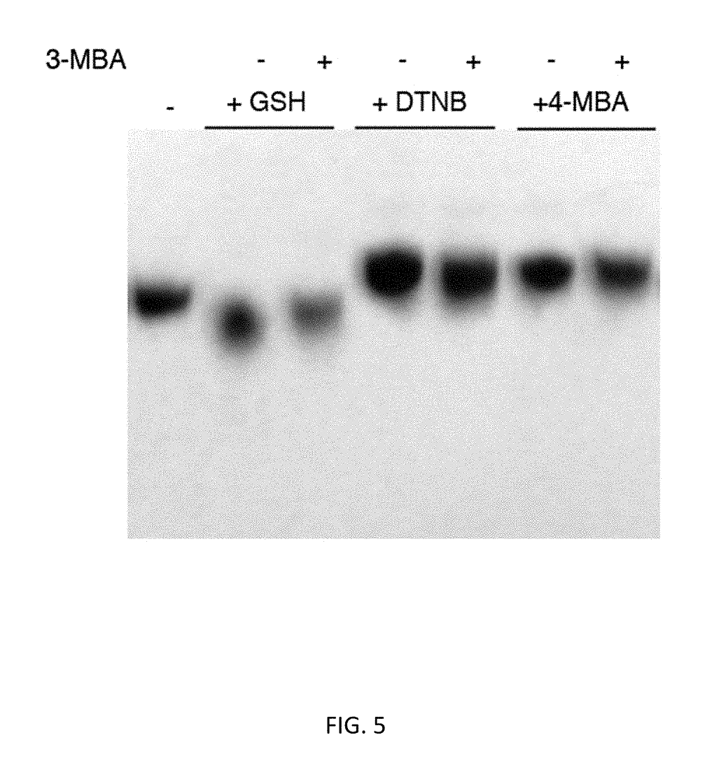

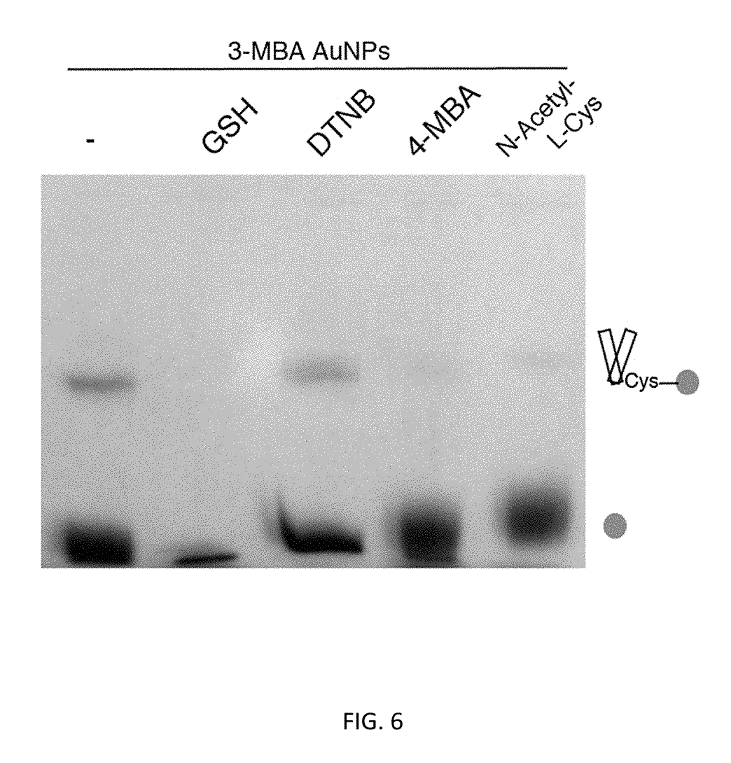

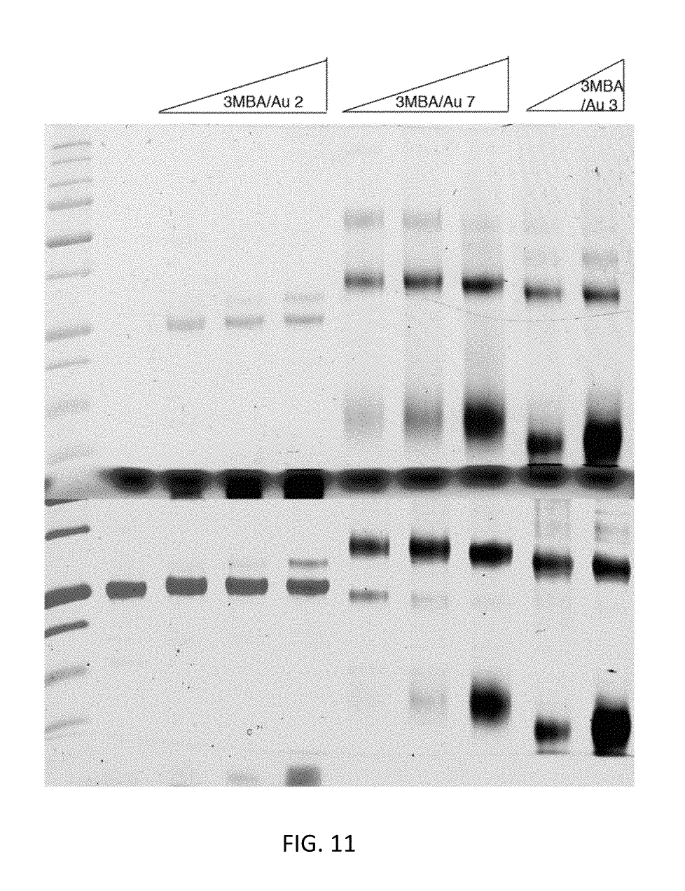

Place exchange showed an order of reactivity, with 3-MBA replaced by every thiol tested but not the reverse (FIG. 5 and FIG. 10). In particular, 3-MBA was replaced by a single chain antibody fragment with a surface-exposed cysteine residue (FIG. 11). Reactivity of 3-MBA-protected particles towards other thiols, including protein sulfhydryl groups, was quenched when 3-MBA was replaced with glutathione (FIG. 6).

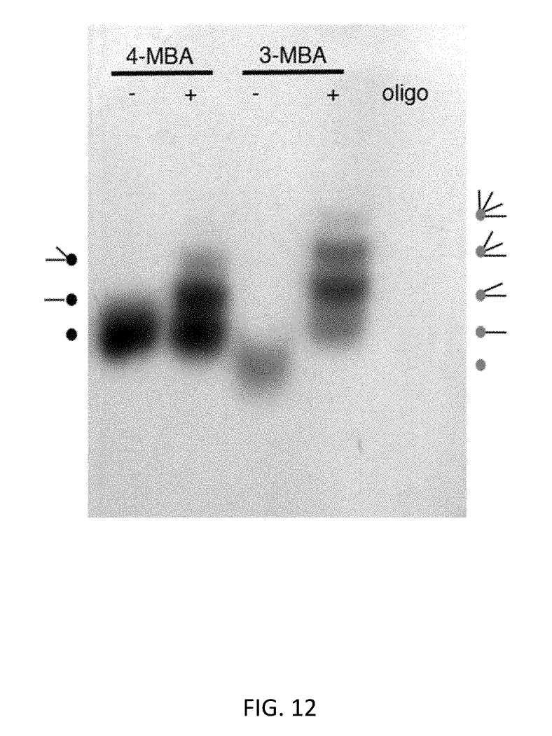

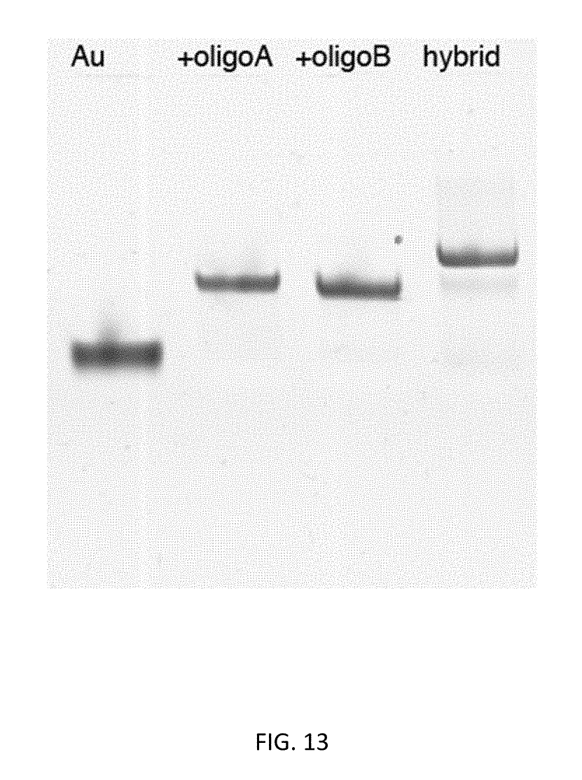

Reactivity of a sulfhydryl group at the 3′-end of an oligonucleotide towards a 3-MBA-protected particle was also greater than towards a 4-MBA-protected particle, as evidenced by the fraction of particles converted to oligonucleotide adducts, and the fraction of particles acquiring multiple oligonucleotides (FIG. 12). A 1:1 conjugate of an oligonucleotide with a 3-MBA-protected particle was isolated by gel electrophoresis and hybridized with the complementary oligonucleotide (FIG. 7S). Hybridization involved boiling the mixture, which had no adverse effect on the oligonucleotide-nanoparticle conjugate.

Summary and Conclusion

Water-soluble gold nanoparticles uniform in size can be synthesized by equilibration of HAuCl₄-thiol solution prior to NaBH₄ reduction. Different sizes are obtained with different Au:thiol ratios. The choice of thiol determines the reactivity of the particles. Reactions of particles with proteins and DNA are described.

Although preferred embodiments of the subject invention have been described in some detail, it is understood that obvious variations can be made without departing from the spirit and the scope of the invention as defined herein.

What is claimed is:

1. A method of synthesizing gold nanoparticles, the method comprising: adding a thiol and chloroauric acid to a mixture of methanol and water, adjusting pH of the mixture to about 13-14, equilibrating the mixture for at least 14 hours, and reacting with borohydride, whereby gold nanoparticles of uniform size are produced.

2. The method of claim 1, wherein the thiol is selected from the group consisting of 3-mercaptobenzoic acid (3-MBA), 4-mercaptobenzoic acid (4-MBA), thiomalate, glutathione, and N-acetyl-L-cysteine.

3. The method of claim 1, wherein the thiol and the chloroauric acid are added at a thiol to gold ratio of at least 2:1.

4. The method of claim 3, wherein the thiol and the chloroauric acid are added at a thiol to gold ratio of at least 3:1.

5. The method of claim 3, wherein the thiol and the chloroauric acid are added at a thiol to gold ratio ranging from 2:1 to 7:1.

6. The method of claim 3, wherein the size of the gold nanoparticles that are produced increases as the thiol to gold ratio is increased.

7. The method of claim 1, further comprising conjugating a gold nanoparticle to a molecule comprising a sulfhydryl group.

8. The method of claim 1, further comprising conjugating a gold nanoparticle to one or more biomolecules.

9. The method of claim 8, wherein the one or more biomolecules are selected from the group consisting of a nucleic acid, an oligonucleotide, a protein, a peptide, a carbohydrate, and a lipid.

10. The method of claim 1, further comprising conjugating a gold nanoparticle to one or more therapeutic agents.

11. The method of claim 1, further comprising conjugating a gold nanoparticle to a targeting agent.

12. The method of claim 1, wherein the mixture is equilibrated for a time ranging from about 16 to about 20 hours.

13. A composition comprising gold nanoparticles of uniform size produced by the method of claim 1.

14. A composition comprising gold nanoparticles of uniform size produced by the method of claim 7.

15. A composition comprising gold nanoparticles of uniform size produced by the method of claim 8.

16. A composition comprising gold nanoparticles of uniform size produced by the method of claim 10.

17. The composition of claim 16, further comprising a pharmaceutically acceptable carrier.

18. A method of treating a disease or disorder comprising administering the composition of claim 17 to a patient in need of treatment for said disease or disorder.

19. A composition comprising gold nanoparticles of uniform size produced by the method of claim 11.

20. A method of imaging gold nanoparticles comprising:
a) administering the composition of claim 19 to a subject, wherein the targeting agent localizes the gold nanoparticles to a site of interest in the subject; and
b) obtaining an image of the gold nanoparticles.

21. The method of claim 20, wherein the gold nanoparticles are further conjugated to a therapeutic agent capable of treating a disease or condition at the site of interest.


SEQUENCE LISTING (1 sequence)

SEQ ID NO: 1 - DNA, 50 bp, Artificial Sequence
  conjugated oligodeoxyribonucleotide
  source (1)..(50): sequence is synthesized
cagatatataaatgcaaaaactgcataaccactttaactaatactttcaa